(19) United States
(12) Patent Application Publication
(10) Pub. No.: US 2026/0078183 A1
(43) Pub. Date: Mar. 19, 2026

(54) TREATING CHRONIC INFLAMMATION AND CANCER BY MACROPHAGE POLARIZATION

(71) Applicant: GEORGIA STATE UNIVERSITY RESEARCH FOUNDATION, INC., Atlanta, GA (US)

(72) Inventors: Zhi-Ren LIU, Atlanta, GA (US); Guangda PENG, Atlanta, GA (US)

(21) Appl. No.: 19/106,336

(22) PCT Filed: Aug. 28, 2023
(86) PCT No.: PCT/US2023/073020

§ 371 Date: Feb. 25, 2025

Related U.S. Application Data
(60) Provisional application No. 63/373,654, filed on Aug. 26, 2022.

(51) Int. Cl. C07K 16/28; A61K 38/45; A61P 35/00; C07K 16/40

(52) U.S. Cl. CPC C07K 16/2818; A61K 38/45; A61P 35/00; C07K 16/40; C12Y 207/0104; C07K 2317/76

(57) ABSTRACT

A method for treating a disease or disorder includes administering to a subject an effective amount of an agent that promotes the polarization of macrophages. The macrophages selectively repolarize their phenotype in the microenvironment. The agent can be PKM2 or mutant thereof. Further, wherein administration of the agent reduces inflammation in the subject.

### BACKGROUND

Tumor progression and metastasis are major contributors to the death of cancer patients. Intrinsic alterations in tumor cells and the crosstalk between cancer cells and their altered microenvironment components promote tumor progression and metastasis. Macrophages populating the surrounding tumor microenvironment (TME) are usually termed as tumor-associated macrophages (TAMs). TAMs create an immunosuppressive TME by producing cytokines, chemokines, growth factors, and triggering the release of inhibitory immune checkpoint proteins in T-cells. Blood monocytes derived from bone marrow hematopoietic stem cells are likely the primary source of macrophages.

Macrophages can display very different and opposing phenotypes, depending on the microenvironment. A macrophage can be activated into different polarized states or two extreme phenotypes: classically activated M1 and alternatively activated M2. M1 macrophages foster an inflammatory response against invading pathogens and tumor cells, whereas M2 macrophages exhibit an immune suppressive phenotype, resulting in tissue repair and tumor progression. M1 macrophages secrete proinflammatory cytokines such as IL-12, tumor necrosis factor (TNF)-α, CXCL-10, and interferon (IFN)-γ, and produce high levels of nitric oxide synthase (NOS, an enzyme metabolizing arginine into the “killer” molecule nitric oxide), while M2 macrophages secrete anti-inflammatory cytokines such as IL-10, IL-13, and IL-4 and express abundant arginase-1, mannose receptor (MR, CD206), and scavenger receptors.

The interchange between M1 (inflammation) and M2 (pro-tumorigenesis or healing) is a biological process termed “macrophage polarization” in response to microenvironmental signals. Macrophages are the most abundant immune cells in a tumor, and their levels directly correlate with cancer progression. TAMs mostly adopt M1 at the early stages of cancer development and predominantly adopt M2 in late-stage cancer. It is well documented that cancer cells educate TAMs toward an M2 phenotype to favor cancer progression, metastasis, and resistance to apoptosis. Abundant M2 macrophages also facilitate cancer relapse after the eradication of tumors by chemotherapies and/or radiation therapies.

Macrophage activation and polarity play roles in modulating inflammation, tissue homeostasis and regeneration, and the resolution of inflammation, with M1 macrophages as pro-inflammatory and M2 macrophages as anti-inflammatory or pro-repair/regeneration. Evidence has suggested that factors causing inflammatory diseases tip the balance of M1/M2 macrophages in favor of M1, often leading to chronic inflammation in tissues/organs and the progression of inflammatory diseases, such as diabetes, colitis, autoimmune diseases, and atherosclerosis.

Accordingly, there is always a need for treatments that modulate or regulate the balance of M1/M2 macrophage polarization. It is to this need, among others, that this application is directed.

### SUMMARY

The application discloses methods for modulating macrophage polarity in a subject in need thereof. These methods include a technique to shift macrophage polarity toward the M2 phenotype, involving the administration of an effective amount of dimeric pyruvate kinase M2 (PKM2) or its mutants that maintain their dimeric form. Alternatively, the method involves promoting macrophage polarity toward the M1 phenotype by administering an effective amount of an antibody with specificity for disrupting the interaction between PKM2 and integrin-αvβ3.

The choice of the specific agent to modulate the PKM2 and integrin-αvβ3 interaction can depend on the condition, disease, or disorder being treated. For instance, if the goal is to reduce inflammation and facilitate processes like angiogenesis and tissue repair or promote the macrophage M2 phenotype, specific embodiments may involve the use of an extracellular, dimeric PKM2 or PKM2 mutants. Conversely, if the aim is to increase inflammatory macrophages or classically activated macrophages, promoting the M1 phenotype in the tumor microenvironment (TME), specific embodiments might include an agent known to disrupt PKM2 and integrin □v□3 interactions, as this is known to enhance tumor cell elimination. In certain diseases, such as those that evade the body's immune response, it can be therapeutically beneficial to induce macrophages to adopt the M1 state rather than the M2 state. Conversely, in diseases resulting from the body's immune response against itself, promoting the M2 state rather than the M1 state can be therapeutic. Hence, improving the condition of a subject with a disease or disorder can be achieved by increasing or decreasing the M2 or M1 polarity of macrophages in the subject.

In another embodiment, an agent that drives macrophage polarity toward the M1 phenotype can be administered alongside an immune checkpoint inhibitor or immune response modifier. A checkpoint inhibitor is a therapeutic agent that stimulates immune cell activity by reducing immunosuppressive checkpoint pathways. The method for treating a disease or disorder involves administering an effective amount of an agent that promotes macrophage polarization, leading to selective repolarization of the macrophage phenotype in the microenvironment. This agent can be PKM2 or a mutant form of PKM2 that preferably adopts a dimeric state, or it can be an antibody targeting PKM2.

In another embodiment, the administration of the agent reduces inflammation in the subject by disrupting PKM2 and integrin αᵥβ₃ interaction. The diseases or disorders that can benefit from selectively repolarizing macrophages from an M1 to an M2 phenotype are those where the effective amount of the agent is the maximum tolerable dose for the subject.

In another embodiment, subjects with diseases or disorders that can benefit from selectively repolarizing macrophages from an M1 to an M2 phenotype may suffer from inflammatory diseases or autoimmune diseases. These diseases can encompass pulmonary diseases, diabetes, cancer, colitis, atherosclerosis, or myocardial infarction.

In another embodiment, a method for treating a disease associated with inflammation in a subject involves administering an effective amount of a composition comprising a pyruvate kinase isoform M2 (PKM2) antibody or binding molecules. This effective amount induces polarization of M1 pro-inflammatory macrophage in the subject, resulting in a macrophage-mediated anti-inflammatory response. The subject can also be given an effective amount of a pyruvate kinase M2 (PKM2) antibody. The method can include the administration of a checkpoint inhibitor, such as pembrolizumab (Keytruda), ipilimumab (Yervoy), nivolumab (Opdivo), and atezolizumab (Tecentriq).

### BRIEF DESCRIPTION OF THE FIGURES

FIG. 1 PKM2 promotes M2 macrophage polarity) shows (A) & (D) Raw 264.7 cells and bone marrow macrophages (BMMj) isolated from healthy mice were treated with indicated agents and then stained by crystal violet. M2 like cells were identified as spread filopodia shaped cells. LPS is a classical inducer of M1 macrophages. IL4 is a classical inducer of M2 macrophages. (B) The level of IL-10 cytokine was measured with ELISA assay using culture media of Raw264.7 treated with indicated agents. (C) Raw264.7 cells were treated with rPKM2 or IL4 and subjected to FACS analysis for CD206+ macrophages. CD206 is a marker for M2 macrophages. (F) & (G) TNFa secretion (typical from M1 macrophages) from Raw 264.7 cells (F) and BMMj (G) was analyzed by ELISA. The cells were treated with indicated agents.

FIG. 2 (PKM2 neutralizing antibody PKM2Ab inhibits tumor growth and lung metastasis) shows (A) 4T1 tumor weight after treatment via indicated agents; (B) & (C) Representative images of HE staining of (B) and quantifications of metastatic nodule number in lung tissues collected from 4T1 mice treated with indicated agents; 4T1 mice were treated: IgG/PKM2Ab 6 mg/kg one dose every two days for 3 weeks, PKM2 wild type (PK2WT), PKM1 (PK1), and PKM2 G415R mutant (G415R), 4 mg/kg one dose every two days for 20 days.

FIG. 3 (PKM2 facilitates M2 macrophage polarity in 4 T1 tumor) shows (A) Representative flow cytometric plots of SSC/CD206 in the metastatic lungs of 4T1 mice treated with indicated agents. (B)-(E) Quantification of FACS analysis of CD206+ (M2 macrophages, right panels) or CD4+FoxP3+ T-reg cell (left panels) population (presented as fold changes of M2 macrophage percentage or T-reg cell percentage). The 4T1 mice were treated with rabbit IgG or PKM2Ab (B and C) or rPKM1/rPKM2 (D and E). FACS analyses were performed with either lung tissue (B and D) or tumor tissues (C and E) of treated 4T1 mice.

FIG. 4 (Extracellular PKM2 facilitates M2 macrophage polarity in B16 melanoma tumor) shows CD206+ (M2 macrophages, right panels) or CD4+FoxP3+ T-reg cell (left panels) population (presented as fold changes of M2 macrophage percentage or T-reg cell percentage) were quantified by FACS analyses. The B16 mice were treated with rPKM1/rPKM2 (A and B) or rabbit IgG or PKM2Ab (C and D). FACS analyses were performed with either tumor tissues (B and D) or lung tissue (A and C) of treated B16 mice.

FIG. 5 (Extracellular PKM2 facilitates M2 macrophage polarity in genetically engineered mouse (GEM) NSCLC tumor) shows CD206+ (M2 macrophages, right panels) or CD4+FoxP3+ T-reg cell (left panels) population (presented as fold changes of M2 macrophage percentage or T-reg cell percentage) were quantified by FACS analyses. The GEM NSCLC mice were treated with rPKM1/rPKM2 (A and B) or rabbit IgG or PKM2Ab (C and D). FACS analyses were performed with tumor tissues collected from the treated mice.

FIG. 6 shows a Kaplan-Meier survival plots (n=14-17) of 4T1 tumor mice (A) and B16 tumor mice (B) treated with indicated agents (PTX, paclitaxel. PD1 antibody against mouse PD-1). Median survival (days) for 4T1 buffer=21, PTX=21, PKM2Ab=27, PKM2Ab+PTX=29 (A), Median survival (days) for B16 IgG=19, PKM2Ab=25, PD1=24, PKM2Ab+PD1=30 (B).

FIG. 7 (Extracellular PKM2 facilitates M2 macrophage polarity in bleomycin induced lung inflammation-fibrosis model) shows (A) & (B) Representative flow cytometric plots of SSC/CD206 (M2 macrophages) in the lung tissues collected from bleomycin-induced lung inflammation-fibrosis mice treated with rPKM1/rPKM2 (A) or IgG/PKM2 antibody (B). (C) & (D) Quantification of CD206+ M2 macrophages in the FACS analyses of the lung tissues from bleomycin induced lung inflammation-fibrosis mice treated rPKM1/rPKM2 (C) or IgG/PKM2 antibody (D). M2 population is presented as fold changes of M2 macrophage percentage.

### DEFINITIONS

The term “administration” refers to providing or delivering a therapeutic agent (e.g., an agent as described herein) to a subject by any effective route. Exemplary routes of administration are described below.

An “autoimmune disease” is a disease or disorder that arises from and is directed against an individual's own tissues. Examples of autoimmune diseases include, but are not limited to Addison's Disease, Allergy, Alopecia Areata, Alzheimer's disease, Antineutrophil cytoplasmic antibodies (ANCA)-associated vasculitis, Ankylosing Spondylitis, Antiphospholipid Syndrome (Hughes Syndrome), arthritis, Asthma, Atherosclerosis, Atherosclerotic plaque, autoimmune diseases (e.g., lupus, RA, MS, Graves' disease, etc.), Autoimmune Hemolytic Anemia, Autoimmune Hepatitis, Autoimmune inner ear disease, Autoimmune Lymphoproliferative syndrome, Autoimmune Myocarditis, Autoimmune Oophoritis, Autoimmune Orchitis, Azoospermia, Behcet's Disease, Berger's Disease, Bullous Pemphigoid, Cardiomyopathy, Cardiovascular disease, Celiac Sprue/Coeliac disease, Chronic Fatigue Immune Dysfunction Syndrome (CFIDS), Chronic idiopathic polyneuritis, Chronic Inflammatory Demyelinating Polyradicalneuropathy (CIDP), Chronic relapsing polyneuropathy (Guillain-Barre syndrome), Churg-Strauss Syndrome (CSS), Cicatricial Pemphigoid, Cold Agglutinin Disease (CAD), chronic obstructive pulmonary disease (COPD), CREST syndrome, Crohn's disease, Dermatitis, Herpetiformis, Dermatomyositis, diabetes, Discoid Lupus, Eczema, Epidermolysis bullosa acquisita, Essential Mixed Cryoglobulinemia, Evan's Syndrome, Exopthalmos, Fibromyalgia, Goodpasture's Syndrome, Hashimoto's Thyroiditis, Idiopathic Pulmonary Fibrosis, Idiopathic Thrombocytopenia Purpura (ITP), IgA Nephropathy, immunoproliferative diseases or disorders (e.g., psoriasis), Inflammatory bowel disease (IBD), including Crohn's disease and ulcerative colitis, Insulin-Dependent Diabetes Mellitus (IDDM), Interstitial lung disease, juvenile diabetes, Juvenile Arthritis, juvenile idiopathic arthritis (JIA), Kawasaki's Disease, Lambert-Eaton Myasthenic Syndrome, Lichen Planus, lupus, Lupus Nephritis, Lymphoscytic Lypophisitis, Meniere's Disease, Miller Fish Syndrome/acute disseminated encephalomyeloradiculopathy, Mixed Connective Tissue Disease, Multiple Sclerosis (MS), muscular rheumatism, Myalgic encephalomyelitis (ME), Myasthenia Gravis, Ocular Inflammation, Pemphigus Foliaceus, Pemphigus Vulgaris, Pernicious Anemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune cholangiopathy, Psoriasis, Psoriatic arthritis, Raynaud's Phenomenon, Reiter's Syndrome/Reactive arthritis, Restenosis, Rheumatic Fever, rheumatic diseases, Rheumatoid Arthritis, Sarcoidosis, Schmidt's syndrome, Scleroderma, Sjorgen's Syndrome, Stiff-Man Syndrome, Systemic Lupus Erythematosus (SLE), systemic scleroderma, Takayasu Arteritis, Temporal Arteritis/Giant Cell Arteritis, Thyroiditis, Type 1 diabetes, Type 2 diabetes, Ulcerative colitis, Uveitis, Vasculitis, Vitiligo, and Wegener's Granulomatosis.

The term “cancer” refers to a condition characterized by unregulated or abnormal cell growth. The terms “cancer cell,” “tumor cell,” and “tumor” refer to abnormal cells or a mass of cells that result from excessive division, which may be malignant or benign, and encompass all pre-cancerous and cancerous cells and tissues.

The term “modulate” or “modulating” generally refers to the ability to alter, by increasing or decreasing, directly or indirectly promoting/stimulating/up-regulating or interfering with/inhibiting/down-regulating a specific concentration or level, such as acting as an antagonist or agonist.

The term “Maximum Tolerable Dose” (MTD), also known as the “Maximum Tolerated Dose” or the “Maximally Tolerated Dose,” is defined as the dose that produces an “acceptable” level of toxicity or that, if exceeded, would put animals or patients at “unacceptable” risk of toxicity. Besides determining animal toxicology, establishing the MTD is the primary objective of Phase I clinical trials, primarily in cancer and HIV treatment, where relatively high doses of drugs are often chosen to achieve the greatest possible beneficial antitumor effect.

The term “polarization” refers to the process by which cells adopt asymmetrical phenotypes in their structure, organization of internal components, or functions, enabling them to perform specific biological functions in organized tissues/organs. Macrophage polarization refers to the ability of macrophages to adopt distinct functional phenotypes in response to signals in the microenvironment. Macrophages can shift between states known as M1 and M2. M1, or classically activated, macrophages secrete pro-inflammatory cytokines (e.g., IL-12, TNF, IL-6, IL-8, IL-1B, MCP-1, and CCL2), are highly phagocytic, and respond to pathogens and environmental insults. M1 macrophages can also be identified by the expression of Nos2. M2, or alternatively activated, macrophages secrete a different set of cytokines (e.g., IL-10) and are generally considered anti-inflammatory. Cells become polarized in response to external cues, such as cytokines, pathogens, injury, and other signals in the tissue microenvironment.

### DETAILED DESCRIPTION

This application discloses methods of regulating M1/M2 macrophage polarization and use of same in therapy. One embodiment includes a method of treating a disease or disorder by increasing or decreasing an M2/M1 macrophage polarization in a subject in need thereof. The TME or the microenvironment can affect macrophage polarization. The administration of an agent that is an extracellular, PKM2 or mutant thereof that equilibrates towards a dimer form promotes M2 macrophages, whereas the administration of an agent that disrupts the PKM2 and integrin promote M1 phenotype macrophages. One aspect is a method of treating an inflammatory disease or disorder and another aspect is a method for treating cancer.

More specifically, the effect of PKM2 on inflammation is supported by findings regarding PKM2's influence on macrophage polarization. Macrophages can be phenotypically polarized by the microenvironment to mount specific functional programs. Polarized Macrophages can be broadly classified into two main groups: 1) classically activated “killer” macrophages (M1), whose prototypical activating stimuli are interferon gamma (INF-γ) and lipopolysaccharides (LPS); and 2) alternatively activated “repair” macrophages (M2) that function in constructive processes like wound healing and tissue repair.

The selection of the specific agent that affects macrophage polarization can depend on the diseases or disorder being treated by the practitioner. If the intent is to reduce inflammation and aid in the process of angiogenesis and tissue repair or promote the M2 phenotype macrophage, specific embodiments can include an agent that interacts with integrin αvβ3 with high affinity, for example, PKM2 or PKM2 mutants that have higher percentage of dimers (e.g., greater than 40%). Certain mutants of PKM2 equilibrate more or less towards a dimer and those that equilibrate more towards the dimer form are more effective. PKM2 can be released into the extracellular space. If the intent is to increase inflammatory macrophages or classically activated macrophages or promote the M1 phenotype, for example, in the TME Specific embodiments can include an agent that reduces the expression of PKM2 or that disrupts PKM2 and integrin αvβ3 interactions with specificity. In some diseases, for example, diseases that evade the body's immune response, it can be therapeutic to allow or cause the macrophages to adopt the M1 state rather than M2 state. In other diseases, for example, diseases that result from the body's immune response to itself, it is therapeutic to allow or cause the macrophages to adopt the M2 state rather than the M1 state. Thus, the condition of a subject with a disease or disorder can be improved by increasing or decreasing the M2/M1 macrophage polarization in the microenvironment or TME of the subject.

### Promote M1 Phenotype

Specific embodiments include a cancer therapy is to stimulate the immune system to kill or destroy cancer cells. Cancer therapy can include the polarization of macrophages towards a pro-inflammatory response (M1), thus allowing the macrophages and other immune cells to destroy the tumor. In one embodiment, the M2 phenotype is driven toward the tumoricidal, or killer, M1 phenotype, thus inhibiting or reducing TAMs' supportive roles in tumors. M1 pro-inflammatory macrophages or classically activated macrophages are aggressive, highly phagocytic, and produce large amounts of reactive oxygen and nitrogen species. Methods for driving macrophages towards a M1-type (macrophage M1 polarization) can include administering to the subject an effective amount of PKM2 binding molecules or other molecules that disrupt the PKM2 integrin αvβ3 interaction (M1-promoting agents). The promotion of macrophage polarization comprises inhibiting or reducing the amount of the repair or healing M2 phenotype in the monocytes and/or macrophages.

One embodiment includes a method of modulating macrophage polarization to kill or destroy cancer cells at a site in a subject by contacting cells or tumor cells with an effective amount of an agent that is a PKM2 binding molecule that disrupts PKM2 and integrin αvβ3 interaction. In this embodiment, the PKM2 binding molecule can be administered to a subject. The agent can be administered in combination with standard chemotherapeutics. In exemplary cancer treatment embodiments, the maximum tolerable dose is given to the subject.

In some embodiments, a PKM2 binding molecule can be administered in combination with a therapy that enhances an immune response, e.g., a checkpoint blockade inhibitor. Exemplary checkpoint inhibitor or immune response modifier can include a pembrolizumab (Keytruda), ipilimumab (Yervoy), nivolumab (Opdivo), atezolizumab (Tecentriq) or Imiquimod. In some embodiment, the PKM2 may be in the extracellular space.

In one specific embodiment, the agent is an anti-PKM2 antibody. Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each antibody for its particular antigen. However, the variability may not be evenly distributed through the variable domains of antibodies and may be concentrated in three segments called complementarity determining regions or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the disclosed antibodies contain at least the CDRs necessary to bind PKM2. An antibody disrupting the interaction between PKM2 and integrin is effective in converting M2 macrophages to M1 macrophages in tumors.

In one embodiment of any one method, the antibody of PKM2 is selected from the group consisting of an antibody against PKM2 bind to PKM2 to disrupt PKM2 and integrin αvβ3 interactions with specificity. Anti-PKM2 antibodies are available commercially and can be developed without undue experimentation. As used herein an antibody can be a polyclonal or a monoclonal antibody that binds PKM2 and is able to disrupt PKM2 and integrin αvβ3 interactions with specificity. For example, the antibody can be a humanized antibody, a chimeric antibody, a Fab, a Fab2, a ScFv, or a single domain antibody. In certain aspects, an antibody of the embodiments comprises a label, such as a radioactive, enzyme, fluorescent or affinity label.

Pharmaceutical compositions containing an agent that disrupts the interaction of PKM2 and integrin αvβ3, for example a PKM2 binding molecule (e.g., anti-PKM2 antibodies) with specificity. Such compositions may be administered in a number of ways to achieve therapeutically effective amounts of the PKM2 binding molecule in the circulation of the subject or the target location in the subject.

Tumor associated M2 polarized macrophages, or TAMs, can effectively suppress T cell proliferation and effector function and promote tumor growth. Reversion of TAMs back to an M1 phenotype has also been reported using an antibody which depletes macrophages directed against the CSF-1 receptor. These approaches have shown limited success in clinic trials due to a narrow therapeutic window and lack of specificity due to targeting all macrophages and not just the aberrant M2 macrophages as intended. Such wholesale depletion of macrophages would be expected to result in increased infection risk and other safety concerns. This method more selectively target the M2 macrophages due to natural tropism for macrophages and tips the balance of function towards the desired M1 phenotype. Thus, in one embodiment, the method of treating cancer does not include use of an antibody which depletes macrophages directed against the CSF-1 receptor.

PKM2 can increase cancer progression by modulating tumor stroma and immunity. PKM2 promotes alternative activated macrophage (M2) in tumors, and PKM2 promotes the M2 macrophage phenotype by interacting with integrin αᵥβ₃ on macrophages and consequently activating integrin signaling. In specific examples, disruption of PKM2 and integrin αᵥβ₃ interactions by a PKM2 antibody abolished the effects of PKM2 on promoting M2 macrophage polarity and increasing the M1 macrophage population in tumors and cancerous tissues/organs.

### Promote M2 Phenotype

M2 phenotype macrophages are anti-inflammatory and can aid in the process of angiogenesis and tissue repair. An inflammatory disease or disorder, e.g., a condition, is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions. The treatment can used to treat patients exhibiting signs of damage to the heart due to, for example, cardiotoxicity, hypertension, valvular disorders, myocardial infarction, viral myocarditis, or scleroderma. A subject can be identified as having or be at risk of having an inflammatory disease or disorder by a skilled practitioner.

One embodiment includes a method of macrophage polarization to treat inflammation associated autoimmune disease in a subject by administering an effective amount of an agent that is PKM2 or mutant thereof. Mutants for use in a method of promoting M2 polarity of macrophages are those that preferentially (or are more preferred from an equilibrium standpoint) adopt the dimer structure present in wt PKM2 or maintain the ability of the mutant to interact with integrin αvβ3 in a functionally similar manner to wt PKM2 (M2-promoting mutants). The method can be used to reduce or ameliorate an autoimmune diseases response. The PKM2 or the M2-promoting PKM2 mutant can be given as or as part of treatment of autoimmune diseases or other diseases associated with inflammation. The application provides methods of reducing or preventing an immune response or immune cell activation in a subject or in isolated immune cells. The efficacy of an agent that inhibits inflammatory or autoimmune diseases or disorders can additionally be assessed using methods described herein. PKM2 can be in the extracellular space and promote the M2 phenotype.

Another aspect provided herein are methods of inhibiting macrophage activation, comprising administering to a subject in need thereof an effective amount of an agent that promotes the M2 phenotype. Whether macrophage activation has occurred can be assessed using standard techniques. For example, by assessing the presence of receptors found on an activated macrophage (e.g., TLR receptors, scavenger receptors, or Fc or complement receptors) or cytokines secreted from activated macrophages. In one embodiment, macrophage activation can be decreased by at least 5, 10, 15, 20, 25, 30, 35, 40, 45, 50, 55, 60, 65, 70, 75, 80, 85, 90, 95, 99%, or more following administration of an agent that increases the interaction of PKM2 and integrin αvβ3, for example, recombinant PKM2 or M2-promoting PKM2 mutant, as compared macrophage activation in an untreated control population.

While PKM2 exists in both a dimeric and tetrameric state, the biologically active protein has a dimeric state and more effective mutants have a higher percentage for the dimeric state at equilibrium. The M2 pyruvate kinase (PKM2), which catalyzes the last but pace-making step of glycolysis, i.e. irreversible dephosphorylation of phosphoenolpyruvate (PEP) to pyruvate, has recently been identified as having a role in cancer progression. PKM2 mutants that can adopt dimeric states are shown in the following illustrative references, which are incorporated herein by reference: Gao, X., Mol Cell 2012 Mar. 9;45(5):598-609.; Zhou, Zhifen, et al. “Oncogenic kinase-induced PKM2 tyrosine 105 phosphorylation converts nononcogenic PKM2 to a tumor promoter and induces cancer stem-like cells.” Cancer research 78.9 (2018): 2248-2261.; Li, iScience 23, 101684, Nov. 20, 2020; Liu, Vivian M., et al. “Cancer-associated mutations in human pyruvate kinase M2 impair enzyme activity.” FEBS letters 594.4 (2020): 646-664; Chen, Tsan-Jan, et al. “Mutations in the PKM2 exon-10 region are associated with reduced allostery and increased nuclear translocation. “Communications biology 2.1 (2019): 1-11; Gupta, Vibhor, et al. “Dominant negative mutations affect oligomerization of human pyruvate kinase M2 isozyme and promote cellular growth and polyploidy.” Journal of Biological Chemistry 285.22 (2010): 16864-16873.; Lv, Lei, et al. “Mitogenic and oncogenic stimulation of K433 acetylation promotes PKM2 protein kinase activity and nuclear localization.” Molecular cell 52.3 (2013): 340-352. Other PKM2 mutants can be developed without undue experimentation. In certain embodiment, PKM2 mutants have a least 75%, preferably at least 85%, more preferably at least 90%, 95%, 98%, 99% or higher or any integral value therebetween nucleotide or amino acid residue identity when compared to wild-type PKM2. These PKM2 mutants can allow polarization of macrophages, resulting in the production of the desired spectrum of inflammatory cytokines needed for anti-tumor immune responses. In one example, recombinant PKM2 (rPKM2) can be used in the methods described herein.

### Administration

The precise dose to be employed in the formulation of the agent will also depend on the route of administration, and the seriousness of the disease or disorder, and should be decided according to the judgment of the practitioner and each patient's circumstances. Effective doses may be extrapolated from dose-response curves derived from the in vitro or animal model test systems described herein.

In certain embodiments, the dosage of the M1-promoting agent is in the range (per gram of body weight) of about 10-100 ng, 100-200 ng, 200-300 ng, 300-400 ng, 400-500 ng, 500-600 ng, 600-700 ng, 700-800 ng, 800-900 ng, 900-1000 ng, 1000-1100 ng, 1100-1200 ng, 1200-1300 ng, 1300-1400 ng, 1400-1500 ng, 1500-1600 ng, 1600-1700 ng, 1700-1800 ng, 1800-1900 ng, 1900-2000 ng, 2000-3000 ng, 3000-4000 ng, 4000-5000 ng, or 6000-7000 ng. Typically, mice were given 3000-4000 ng/g or 3-4 mg/kg of the M1-promoting agent. In certain embodiments, the dosage of the M2-promoting agent is in the range (per gram of body weight) of about 10-100 ng, 100-200 ng, 200-300 ng, 300-400 ng, 400-500 ng, 500-600 ng, 600-700 ng, 700-800 ng, 800-900 ng, 900-1000 ng, 1000-1100 ng, 1100-1200 ng, 1200-1300 ng, 1300-1400 ng, 1400-1500 ng, 1500-1600 ng, 1600-1700 ng, 1700-1800 ng, 1800-1900 ng, 1900-2000 ng, 2000-3000 ng, 3000-4000 ng, 4000-5000 ng, 6000-7000 ng, or 7000-8000 ng. Typically, mice were given 2000-5000 ng/g or 2-5 mg/kg of the M2-promoting agent.

In some embodiments, the composition is administered into the same subject by multiple routes of administration. In some embodiments, said multiple routes of administration comprise intravenous administration, intra-arterial administration, intrathecal administration, intranasal administration, intraperitoneal administration, and/or periocular administration. In some examples, the PKM2 agent can be administered intravenously to the circulatory system of the subject. In some examples, the PKM2 agent can be infused in suitable liquid and administered into a vein of the subject.

In some embodiments, the composition is administered to result in extracellular PKM2. In other cases, the level of extracellular PKM2 can be measured and/or observed.

Efficacy testing can be performed during treatment using the methods described herein. Measurements of the degree of severity of several symptoms associated with an ailment are noted prior to the start of a treatment and then at later specific time period after the start of the treatment.

### EXAMPLES

The following examples are provided to offer those with ordinary skill in the art a comprehensive disclosure and description of how to make and use the present invention. They are not intended to limit the scope of what the inventors regard as their invention, nor are they intended to imply that the experiments below are exhaustive or the only experiments conducted.

### Example 1: EcPKM2 Promotes M2 Macrophage

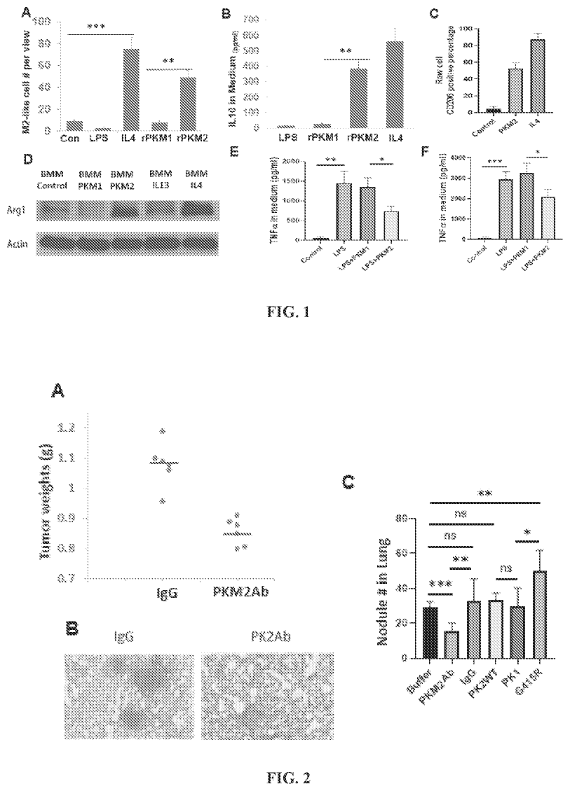

The effects of extracellular PKM2 (EcPKM2) or (ExPKM2) were tested on cultured Raw 264.7 cells and bone marrow macrophages (BMM) isolated from healthy mice. A detailed examination of the morphology of Raw264.7 and BMM cells treated with various agents, including recombinant PKM2 (rPKM2), rPKM1, LPS, or IL-4, revealed that cells treated with rPKM2 resembled IL-4 treated cells in terms of morphology. It is well known that IL-4 activates M2 macrophages, whereas LPS stimulates M1 macrophages. The quantified cell numbers of the adopted M1/M2 macrophage treatment with different agents were obtained. EcPKM2 facilitated M2 macrophages, as shown in FIG. 1A. It is well documented that M2 macrophages secrete IL-10, and these macrophages express the cell surface marker CD206. Thus, Applicant examined the secretion of IL-10 in the culture medium of Raw264.7 cells treated with rPKM2, rPKM1, IL-4, or LPS. Cells treated with the vehicle, rPKM1, or LPS did not secrete IL-10, while cells treated with rPKM2 and IL-4 secreted high levels of IL-10 (FIG. 1B). FACS analysis of Raw264.7 cells treated with the vehicle, rPKM1, rPKM2, or IL-4 indicated that macrophages expressed high levels of CD206 upon IL-4 or rPKM2 treatment, while CD206 expression was almost undetectable in vehicle or rPKM1 treated macrophages (FIG. 1C). The effects of EcPKM2 on macrophage polarity were further analyzed with BMM by immunoblotting, measuring arginase 1 (Arg1), an M2 macrophage marker, levels in the treated cells. Evidently, similar to the effects of IL-4, EcPKM2 increased Arg1 in BMM cells (FIG. 1D). Applicant further tested whether EcPKM2 could convert M1 macrophages to the M2 subtype. To this end, Raw264.7 or BMM cells were first incubated with LPS, and then rPKM2 was added to the cell culture. Interestingly, the addition of rPKM2 shifted LPS-induced M1 macrophages to the M2 phenotype, as measured by the typical M1 secretion cytokine TNFa (FIG. 1 E&F). All the results suggest that EcPKM2 may be capable of converting M1 macrophages to M2 in vitro.

### Example 2: EcPKM2 Increased M2 Macrophages and Decreased M1 Macrophages in Tumors

Cancer cells secrete PKM2, and circulating PKM2 correlates with cancer progression {}. EcPKM2 promotes M2 macrophages in vitro. To test whether EcPKM2 promotes M2 macrophages in vivo, we employed the orthotopic 4T1 tumor model. The mice carrying 4T1 tumors were treated with rPKM2, rPKM1, or a vehicle when tumors grew to an average size of 200 mm in diameter. At the end of the experiments, tumor weights were examined. Clearly, rPKM2, but not rPKM1 and the vehicle, increased tumor weight. FACS analyses of macrophages by F4/80 and CD206 sorting indicated that rPKM2 treatment increased the percentage of M2 macrophages and decreased the percentage of M1 macrophages in 4T1 tumors compared to the rPKM1 group (FIG. 3E). It is well documented that M2 macrophages induce CD4+ Treg cells in tumors. Thus, we analyzed CD4+ Treg cells in rPKM2 or rPKM1 treated 4T1 tumors. The Treg cells increased in tumors of rPKM2 treated 4T1 mice (FIG. 3E). Examination of metastatic nodules in the lung revealed that rPKM2 treatment increased both metastatic nodular number and size compared to the group treated with rPKM1 and the vehicle (FIG. 2C). We asked whether rPKM2 treatment affected macrophage polarity in lung metastatic tumors. FACS analyses showed that rPKM2 increased M2 macrophages in lung metastatic tumors compared to rPKM1 (FIG. 3D). Similarly, rPKM2 treatment also increased Treg cells in lung metastatic tumors (FIG. 3D).

EcPKM2 promotes M2 macrophage by interacting with and activating integrin αᵥβ₃. We reasoned that an antibody disrupting the PKM2 and integrin αᵥβ₃ interaction would exert opposite effects in tumors. We used a developed monoclonal anti-PKM2 antibody (IgGPK) that disrupts the PKM2 and integrin αᵥβ₃ interaction {}. Tumor-bearing mice were treated with IgGPK or rabbit IgG as a control. IgGPK treatment led to smaller tumors and fewer and smaller metastatic nodules in the lung compared to the IgG treatment group (FIG. 2A-C). IgGPK led to a high M1 and low M2 macrophage population in primary tumors (FIG. 3A&C). FACS analyses of Treg in the tumor also demonstrated that IgGPK resulted in a decrease in Treg (FIG. 3C). FACS analyses of M1/M2 macrophage and Treg in lung metastatic tumors revealed that IgGPK treatment decreased M2 macrophages and Treg in tumors, while it increased M1 macrophages (FIG. 3B). Histology analyses showed that IgGPK decreased IL-10 in both primary and metastatic tumors. We conclude from our experiments that EcPKM2 promotes M2 macrophages, while an antibody disrupting the EcPKM2 and integrin αᵥβ₃ interaction abrogates the effects of EcPKM2 on the polarity of TAMs.

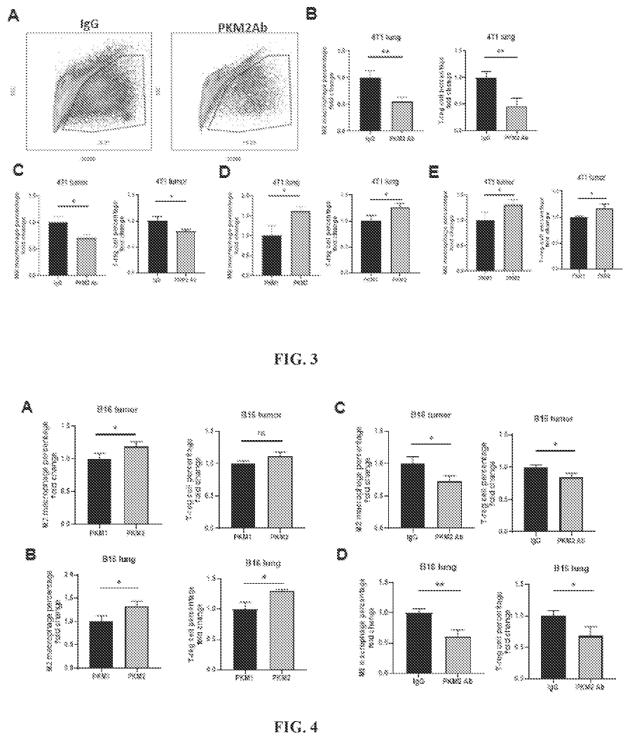

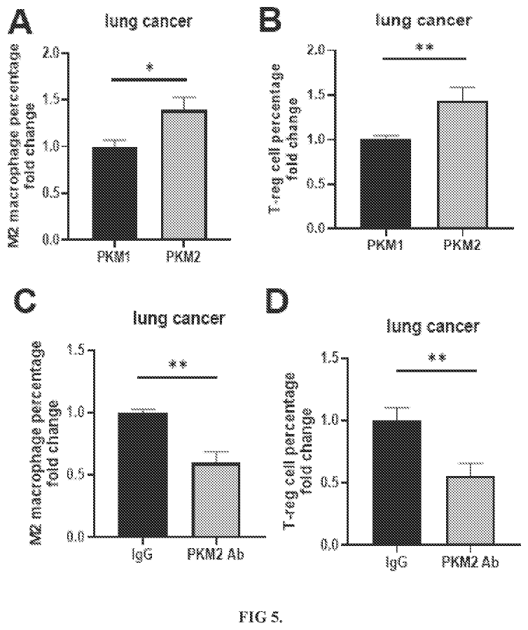

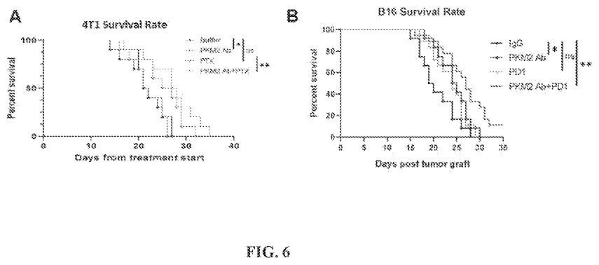

To test the commonality of the role of EcPKM2 in controlling TAM polarity in tumors, we analyzed the effects of rPKM2 with two additional murine cancer models. Orthotopic xenograft of B16 cells, a murine melanoma cell line. DuPage, M., and co-workers developed a genetic engineering mouse (GEM) lung cancer model (KP-NSCLC). The GEM mice spontaneously develop lung cancer at the age of 3 weeks due to the specific expression of KrasG12D and Trp53R172H in the lung. B16 tumor-bearing mice 7 days post-tumor inoculation or 8 weeks old KP-NSCLC mice were treated with rPKM1/rPKM2 or a vehicle. The tumor size in rPKM2 treated mice was larger than that in rPKM1 treated mice. The nodule number in rPKM2 treated KP-NSCLC mice was higher than that in rPKM1 treated mice. Analyses of macrophages and Treg in the tumors by FACS demonstrated that rPKM2 treatment increased M2 macrophages and Treg in tumors of both B16 and KP-NSCLC models (FIG. 4 and FIG. 5). Conversely, IgGPK treatment led to smaller tumors in size and nodule number (FIG. 4 and FIG. 5). Importantly, IgGPK decreased M2 macrophages and Treg and increased M1 macrophages (FIG. 4 and FIG. 5)) in tumors of both models. Examination of macrophage and Treg in lung metastatic tumors of the treated B16 mice showed that rPKM2 increased M2 macrophages and Treg and decreased M1 macrophages in lung metastatic tumors, while IgGPK reduced M2 macrophages and Treg in B16 lung metastatic tumors (FIG. 4 and FIG. 5). These results further confirmed our conclusion that EcPKM2 promotes M2 macrophages in both primary and metastatic tumors, while an antibody that disrupts the interaction between EcPKM2 and integrin αvβ3 promotes M1 macrophages.

### Example 3: IgGPK Enhanced Efficacy of Cancer Chemotherapeutics

It is now recognized that M2 TAMs strongly facilitate chemotherapy resistance and relapse after the eradication of tumors by chemotherapy and/or radiation therapies. An antibody that disrupts EcPKM2 and integrin αvβ3 interaction promotes TAMs M2 to M1 conversion. 4T1 murine breast cancer model was used to test the disruption. Tumor-bearing mice were treated with IgGPK and a combination of IgGPK with a low dose of paclitaxel (PTX). PTX alone did not have a significant effect on overall survival and tumor growth. IgGPK provided modest benefits in terms of animal overall survival and tumor growth. However, the combination prolonged the survival of tumor-bearing mice and inhibited tumor growth (FIG. 6A). Analyses of macrophages in tumors 10 days after treatment by FACS showed that the combination resulted in an increase in M1 macrophages compared to PTX or IgGPK alone.

### Example 4: IgGPK Enhances Efficacy of Checkpoint Blockades

The addition of checkpoint inhibitors to various chemotherapies represents an important advancement in the treatment of many different types of cancers. It is well documented that macrophages play a critical role in modulating cancer immunity by secreting pro-or anti-inflammatory soluble factors, such as cytokines/chemokines. The anti-PKM2 antibody IgGPK that disrupts the interaction between EcPKM2 and integrin αvβ3 promotes M2 macrophages in tumors. It would be intriguing to test the anti-cancer effects of IgGPK in combination with checkpoint blockade. B16 is a murine melanoma model that is well tested with checkpoint blockade treatment. We, therefore, used this model to test the efficacy of a combination of IgGPK+anti-PD-1 antibody (aPD-1). Tumor-bearing mice were treated with IgGPK, aPD-1, and IgGPK+aPD-1. IgGPK and aPD-1 alone provided marginal or modest benefits in terms of animal overall survival and tumor growth. The combination provided anti-cancer effects (FIG. 6B). Tumors in three out of 14 mice completely disappeared and never reappeared.

### Example 5: EcPKM2 Facilitates M2 Macrophage in Lung Chronic Inflammation

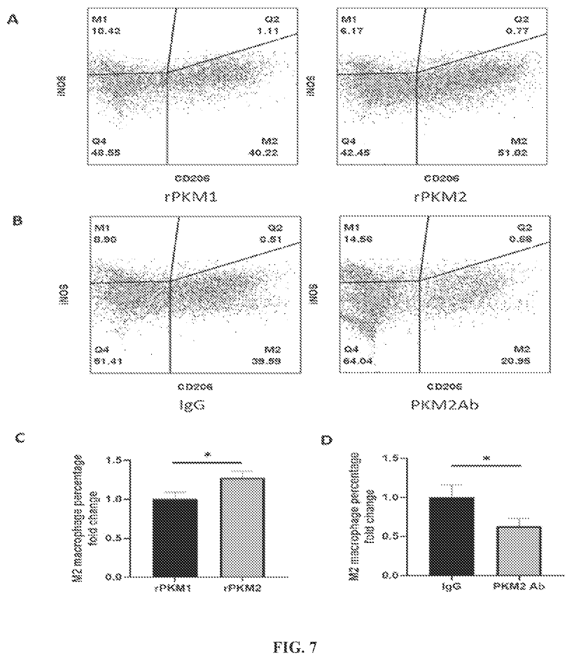

To test the effects of EcPKM2 in modulating inflammation, a bleomycin-induced lung inflammation and fibrosis model was used. C57BL/J mice were induced with inflammation/fibrosis through intraperitoneal (i.p.) administration of bleomycin (25 mg/kg, twice weekly) for 3 weeks, followed by co-administration of bleomycin plus rPKM2/rPKM1 (4 mg/kg) for a total of 8 weeks, or for 6 weeks followed by co-administration of bleomycin plus PKM2Ab/IgG (6 mg/kg) for a total of 9 weeks. Mice were sacrificed one day after the last dose treatment. Analyses of macrophages in the lung tissue by FACS using CD206+ demonstrated that rPKM2 treatment increased M2 macrophages, while PKM2Ab decreased M2 macrophages (FIG. 7).

1. A method for treating a disease or disorder, comprising administering to a subject an effective amount of an agent that promotes the polarization of macrophages, wherein the macrophages selectively repolarize their phenotype in the microenvironment.

2. The method of claim 1, wherein the agent is a PKM2 or a mutant that of PKM2 that preferentially adopts a dimeric state.

3. The method of claim 1, wherein the agent is an antibody to PKM2.

4. The method of claim 1, wherein the administration of the agent reduces inflammation in the subject, and the agent is a PKM2-binding molecule that disrupts the interaction between PKM2 and integrin αvβ3.

5. The method of claim 1, wherein the disease or disorder can benefit from selectively repolarizing the macrophages from an M1 to an M2 phenotype, and the effective amount of the agent is the maximum tolerable dose for the subject.

6. The method of claim 5, wherein the disease or disorder that can benefit from selective repolarization of the macrophages from an M1 to an M2 phenotype is an inflammatory disease or an autoimmune disease.

7. The method of claim 6, wherein the disease or disorder is a pulmonary disease

8. The method of claim 6, wherein the disease or disorder is diabetes.

9. The method of claim 6, wherein the disease or disorder is cancer.

10. The method of claim 6, wherein the disease or disorder is colitis.

11. The method of claim 6, wherein the disease or disorder is atherosclerosis.

12. The method of claim 6, wherein the disease or disorder is myocardial infarction.

13. A method for treating a disease associated with inflammation in a subject, comprising administering to the subject an effective amount of a composition having a pyruvate kinase isoform M2 (PKM2) antibody or binding molecules, wherein the effective amount induces M1 pro-inflammatory macrophage polarization in the subject, and the M1 macrophage polarization results in a macrophage-mediated inflammatory response.

14. The method of claim 13, wherein PKM2 antibody is administered with anti-cancer radiation therapies.

15. The method of claim 13, wherein the PKM2 antibody is a monoclonal antibody that selectively binds the PKM2.

16. A method for macrophage polarization in a subject in need thereof, comprising administering to the subject an effective amount of
(a) a pyruvate kinase M2 (PKM2) antibody, wherein the macrophage polarization is between M1 and M2; or
(b) pyruvate kinase M2 (PKM2) or a mutant thereof, wherein upon contact with PKM2 or the mutant thereof, macrophages polarize from the M2 to the M1 phenotype.

17. (canceled)

18. (canceled)

19. The method of claim 16, wherein the PKM2 or mutant thereof is administered in combination with a checkpoint inhibitor, wherein the checkpoint inhibitor is selected from pembrolizumab (Keytruda), ipilimumab (Yervoy), nivolumab (Opdivo), or atezolizumab (Tecentriq).

20. The method of claim 18, wherein the PKM2 or mutant thereof is applied extracellularly.

21. (canceled)

22. The method of claim 19, wherein PKM2 or a mutant thereof is administered in combination with anti-cancer chemotherapeutics.

23. The method of claim 1, wherein the PKM2 is in the extracellular space.
